(19) United States
(12) Patent Application Publication
(10) Pub. No.: US 2021/0186716 A1
(43) Pub. Date: Jun. 24, 2021

(54) PYRAMIDAL PROSTHETIC FOOT

(71) Applicant: HARIPRIYA SUMANA GOSAKAN, BANGALORE (IN)

(72) Inventor: HARIPRIYA SUMANA GOSAKAN, BANGALORE (IN)

(21) Appl. No.: 17/259,544

(22) PCT Filed: Jul. 11, 2019
(86) PCT No.: PCT/IN2019/050514

§ 371 Date: Jan. 11, 2021

(30) Foreign Application Priority Data
Jul. 13, 2018 (IN) 201841026207

(51) Int. Cl. A61F 2/66; A61F 2/50

(52) U.S. Cl. CPC A61F 2/66; A61F 2/5044; A61F 2002/5055; A61F 2002/6678; A61F 2002/6671

(57) ABSTRACT

A prosthetic foot comprises a hollow triangular lightweight top section that is an integral part of a first leafspring that originates from a top portion to the toe of the prosthetic foot. The prosthetic foot further comprises a second leaf spring. The second leaf spring originates from the heel to the toe.

### CROSS-REFERENCE TO RELATED APPLICATIONS

This PCT application claims priority to and the benefit of provisional patent application titled “Pyramidal Prosthetic Foot”, application number 201841026207, filed in the Indian Patent Office on 13 Jul. 2018. The specification of the above referenced patent application is incorporated herein by reference in its entirety.

### BACKGROUND

The apparatus disclosed herein, in general, relates to a medical device, and in particular, relates to a prosthetic foot.

Amputees with a loss of a limb experience discomfort while using heavy prosthetic feet. FIG. 1 (prior art) illustrates a prosthetic foot 101 with a heavy metallic connector 102. The flat vertical composite section of the prosthetic foot 101 has reduced stiffness, and hence requires a large form factor metallic connector 102 to avoid over flexure. There is an unmet need for reducing the weight of metallic connectors and fittings in prosthetic feet.

### SUMMARY OF THE INVENTION

The prosthetic foot disclosed herein addresses the above recited unmet need for reducing the weight of metallic connectors and fittings in prosthetic feet.

### BRIEF DESCRIPTION OF THE DRAWINGS

FIG. 1 (prior art) exemplarily illustrates a prosthetic foot with a heavy metallic connector.

FIG. 2 illustrates a prosthetic foot with a triangular composite support for supporting a lightweight metallic connector.

### DETAILED DESCRIPTION OF THE INVENTION

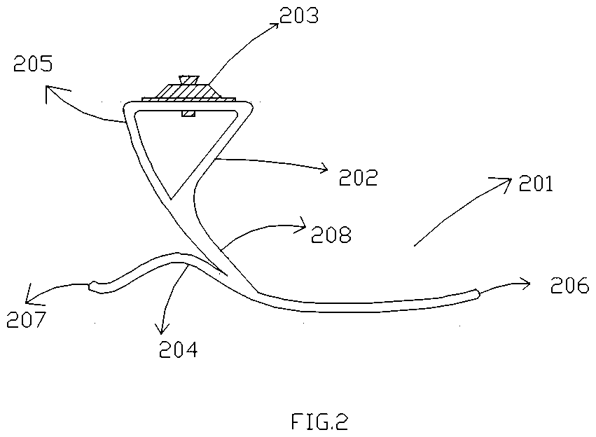

FIG. 2 illustrates a prosthetic foot with a triangular composite support 201 for supporting a lightweight metallic connector 203. The prosthetic foot 201 comprises a hollow thin walled lightweight triangular top section 202 that is an integral part of a first leafspring 208 that originates from a top portion 205 to the toe 206 and a second leaf spring 204 that originates from the heel 207 to the toe 206. Fiber continuity is established between the walls of the triangular composite support 202 by wrapping a prepeg around a triangular mold and removing the mold after curing the prepeg. The prosthetic foot 201 disclosed herein comprises a triangular carbon fiber composite section 202 that supports a small form factor metallic pyramidal connector 203. The stiff triangular shape reduces deflection and does not require a heavy weight metallic connector for added strength. In an embodiment, a carbon fiber composite section of a closed square shape or a circle shape may be used instead of the triangular carbon fiber composite section 202. However, the triangular carbon fiber composite section 202 requires the least amount of material for an optimised stiffness to weight ratio. The prosthetic foot 201 is manufactured using one or a combination of the following materials: carbon fiber composites, glass fiber composites, plastics, metal, etc. For example, the prosthetic foot 201 comprises carbon fiber in a thermosetting or thermoplastic matrix.

For example, unidirectional carbon fiber prepegs may be laid in a multiaxial layup sequence, and high temperature cured in a pressurized vacuum bagged environment in a metallic mold at a high temperature of 150 Celcius.

For example, the triangular carbon fiber composite section 202 may be of thickness in the range 2 mm to 5 mm, and preferably of 3 mm to 4 mm. The fiber layup sequence may comprise the following: Top woven 0/90, +45, −45, 0, 0, 0, 0, 0, 0, 0, 0−45,+45, Lower woven 0/90. The thickness of each prepeg layer may be in the 0.1 mm to 0.25 mm. To increase thickness, the number of prepeg layers may be increased.

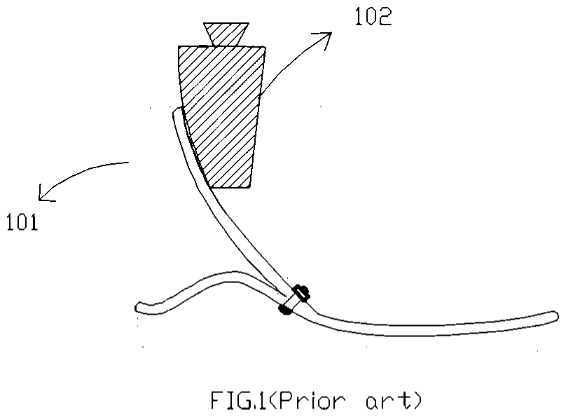

Considerable weight savings are gained by the use of the triangular carbon fiber composite section 202. For example, with a 5 cm walled equilateral triangular section, a weight saving of approximately 140 to 180 gm may be realized in the disclosed triangular section 202 in FIG. 2 when compared to the heavy metallic connector section 102 illustrated in prior art FIG. 1.

The foregoing examples have been provided merely for explanation and are in no way to be construed as limiting of the prosthetic foot 201 disclosed herein. While the prosthetic foot 201 has been described with reference to various embodiments, it is understood that the words, which have been used herein, are words of description and illustration, rather than words of limitation. Furthermore, although the prosthetic foot 201 has been described herein with reference to particular means, materials, and embodiments, the prosthetic foot 201 is not intended to be limited to the particulars disclosed herein; rather, the prosthetic foot 201 extends to all functionally equivalent structures, methods and uses, such as are within the scope of the appended claims. While multiple embodiments are disclosed, it will be understood by those skilled in the art, having the benefit of the teachings of this specification, that the prosthetic foot 201 disclosed herein is capable of modifications and other embodiments may be effected and changes may be made thereto, without departing from the scope and spirit of the prosthetic foot 201 disclosed herein.

I claim:

1. A prosthetic foot comprising:
a hollow triangular lightweight top section that is an integral part of a first leafspring that originates from a top portion to the toe of said prosthetic foot; and
a second leaf spring that originates from the heel to the toe of said prosthetic foot.

2. The prosthetic foot of claim 1, wherein said first composite leafspring and second campsite leafspring further comprise a composite fiber in a resin matrix.

3. The prosthetic foot of claim 2, wherein said composite fiber is one of or a combination of carbon fiber, aramid fiber and glass fiber.

4. The prosthetic foot of claim 2, wherein said resin matrix is one of epoxy, vinyl ester, polyester or polyurethane.
